(19) United States
(12) Patent Application Publication
(10) Pub. No.: US 2019/0125439 A1
(43) Pub. Date: May 2, 2019

(54) USE OF ELECTROMAGNETIC FIELDS IN IRE DEVICE DELIVERY AND THERAPY MONITORING

(71) Applicants: Boston Scientific Scimed Inc., Maple Grove, MN (US); Mayo Foundation for Medical Education and Research, Rochester, MN (US)

(72) Inventors: James P. Rohl, Prescott, WI (US); Sarah M. Gruba, Vadnais Heights, MN (US); Douglas D. Pagoria, Forest Lake, MN (US); Matthew Hein, Eden Prairie, MN (US); Samuel J. Asirvatham, Rochester, MN (US); Chance M. Witt, Rochester, MN (US); Suraj Kapa, Rochester, MN (US)

(21) Appl. No.: 16/170,689

(22) Filed: Oct. 25, 2018

Related U.S. Application Data
(60) Provisional application No. 62/577,539, filed on Oct. 26, 2017.

(51) Int. Cl. A61B 18/14

(52) U.S. Cl. CPC A61B 18/1492; A61B 2018/00613

(57) ABSTRACT

Various aspects of the present disclosure are directed toward apparatuses, systems, and methods for assessing electroporation therapy applied to a tissue region of a patient. In certain instances, the apparatuses, systems, and methods may include one or more sensors configured to sense an application of electroporation energy by an electroporation device to determine a location of the electroporation device within the patient.

### CROSS-REFERENCE TO RELATED APPLICATION

This application claims priority to Provisional Application No. 62/577,539, filed Oct. 26, 2017, which is herein incorporated by reference in its entirety.

### TECHNICAL FIELD

The present disclosure generally relates to systems and methods for providing a therapy to a patient. More particularly, the present disclosure relates to apparatuses, systems, and methods assessing electroporation.

### BACKGROUND

Electroporation may permeate cell membranes through exposure to certain electric pulses. Irreversible electroporation may be alternative for the ablation of undesired tissue. Electroporation without thermal effect to ablate tissue may avoid thermal damage to target tissue or other tissue surrounding the target tissue and/or ablates cells without damaging the blood vessel structure.

Atrial fibrillation is an irregular and often rapid heart rate that commonly causes poor blood flow to the body. Ablation procedures, including ablation of thoracic veins such as the pulmonary vein, may be a treatment for atrial fibrillation. During pulmonary vein ablation, for example, catheters are inserted into the atrium and energy is delivered to the tissue of the pulmonary vein and/or near the ostia of the pulmonary veins in the left atrium. Ablative energy, by electroporation, may be used in other areas of the heart, other veins, or blood vessels.

Analysis and/or tracking of the electroporated tissue may be useful in effectively and accurately ablating tissue using electroporation. Presently, it may take days or months to assess the irreversible nature of the procedure.

### SUMMARY

In Example 1, an apparatus for assessing electroporation therapy applied to a tissue region of a patient, the apparatus including an array of sensors configured to sense an application of electroporation energy by an electroporation device to determine a location of the electroporation device within the patient; and an output device configured to indicate the location of the electroporation device within the patient.

In Example 2, the apparatus of Example 1, wherein the output device is configured to verify non-movement of the electroporation device between pulse bursts of the electroporation device.

In Example 3, the apparatus of any of Examples 1 or 2, wherein the array of sensors are configured to sense an electromagnetic field generated by the electroporation device resulting from pulse bursts applied by the electroporation device.

In Example 4 the apparatus of Example 3, wherein the output device is configured to measure a difference between the electromagnetic field generated by the electroporation device prior to application of the pulse bursts and the electromagnetic field generated by the electroporation device after application of the pulse bursts to determine the location of the electroporation device within the patient.

In Example 5, the apparatus of Example 4, wherein the output device is configured to measure the difference based on a change in cell density at the tissue region.

In Example 6, the apparatus of Example 5, wherein the output device is configured to measure the change in cell density to locate impacted and non-impacted cells in the tissue region.

In Example 7, the apparatus of Example 6, wherein the output device is configured to display an indication of the impacted and non-impacted cells during application of the application of electroporation energy.

In Example 8, the apparatus of any of Examples 1-7, wherein output device is further configured to verify effectiveness of the application of electroporation energy by the electroporation device at the tissue region.

In Example 9, the apparatus of any of Examples 1-8, wherein the array of sensors are at least one of a two-dimension array of magnoresistive sensors and three-dimensional array of magnoresistive sensors.

In Example 10, the apparatus of Example 9, wherein the magnoresistive sensors include at least one of Magneto-Resistance (AMR) sensors, Giant Magneto-Resistance (GMR) sensors, Magnetic Tunneling Junction (MTJ) sensors, Tunnel Magneto-Resistance (TMR) sensors, inductive sensors, fluxgate sensors, GMI (giant magnetoimpedance) sensors, and hall sensors.

In Example 11, the apparatus of any of Examples 1-10, wherein the array of sensors are configured to measure an output magnetic field density emitted by the electroporation device over a grid of the array of sensors.

In Example 12, the apparatus of any of Examples 1-11, wherein the output device is configured to indicate the location of the electroporation device within the patient during the application of electroporation energy by the electroporation device.

In Example 13, the apparatus of any of Examples 1-12, wherein the electroporation device includes a balloon structure and one or more electrodes arranged on or within the balloon structure and configured to deliver energy to the tissue region.

In Example 14, the apparatus of Example 13, wherein the one or more electrodes includes a proximal electrode arranged near a proximal end of the balloon structure and a distal electrode arranged near a distal end of the balloon structure, and the proximal electrode includes portions extending toward the distal electrode and the distal electrode includes portions extending toward the proximal electrode.

In Example 15, the apparatus of Example 14, wherein the proximal electrode and the distal electrode are spaced apart by between 0 mm and 25 mm.

In Example, 16 an apparatus for assessing electroporation therapy applied to a tissue region of a patient, the apparatus including: an array of sensors configured to sense an electromagnetic field generated during the application of electroporation energy by an electroporation device to determine a location of the electroporation device within the patient; and an output device configured to indicate the location of the electroporation device within the patient.

In Example 17, the apparatus of Example 16, wherein the output device is configured to verify non-movement of the electroporation device between pulse bursts of the electroporation device.

In Example 18, the apparatus of Example 16, wherein the output device is configured to measure a difference between in the electromagnetic field generated by the electroporation device prior to application of the pulse bursts and the electromagnetic field generated by the electroporation device after application of the pulse bursts effective cells to determine the location of the electroporation device within the patient.

In Example 19, the apparatus of Example 18, wherein the output device is configured to measure the difference based on a change in cell density at the tissue region.

In Example 20, the apparatus of Example 19, wherein the output device is configured to measure the change in cell density to locate impacted and non-impacted cells in the tissue region.

In Example 21, the apparatus of Example 20, wherein the output device is configured to display an indication of the impacted and non-impacted cells during application of the application of electroporation energy.

In Example 22, the apparatus of Example 16, wherein output device is further configured to verify effectiveness of the application of electroporation energy by the electroporation device at the tissue region.

In Example 23, the apparatus of Example 16, wherein the electroporation device includes a balloon structure and one or more electrodes arranged on or within the balloon structure and configured to deliver energy to the tissue region.

In Example 24, an apparatus for assessing electroporation therapy applied to a tissue region, the apparatus including: an electroporation device having: a catheter sized and shaped for vascular access and including an elongate body extending between a proximal end and a distal end, a balloon structure arranged near the distal end of the elongate body, and one or more electrodes arranged on or within the balloon structure and configured to deliver energy to the tissue region; and an array of sensors configured to sense an application of electroporation energy by the electroporation device to determine a location of the electroporation device within the patient.

In Example 25, the apparatus of Example 24, wherein the array of sensors are at least one of a two-dimension array of magnoresistive sensors and three-dimensional array of magnoresistive sensors.

In Example 26, the apparatus of Example 25, wherein the magnoresistive sensors include at least one of Magneto-Resistance (AMR) sensors, Giant Magneto-Resistance (GMR) sensors, Magnetic Tunneling Junction (MTJ) sensors, and Tunnel Magneto-Resistance (TMR) sensors, inductive sensors, fluxgate sensors, GMI (giant magnetoimpedance) sensors, and hall sensors.

In Example 27, the apparatus of Example 24, wherein the array of sensors are configured to measure an output magnetic field density emitted by the electroporation device over a grid of the array of sensors.

In Example 28, the apparatus of Example 24, wherein the one or more electrodes includes a proximal electrode arranged near a proximal end of the balloon structure and a distal electrode arranged near a distal end of the balloon structure, and the proximal electrode includes portions extending toward the distal electrode and the distal electrode includes portions extending toward the proximal electrode.

In Example 29, the apparatus of Example 28, wherein the proximal electrode and the distal electrode are spaced apart by between 0 mm and 25 mm.

In Example 30, the apparatus of Example 24, wherein output device is further configured to verify effectiveness of the application of electroporation energy by the electroporation device at the tissue region.

In Example 31, a method of assessing electroporation therapy applied to a tissue region of a patient, the method including: applying electroporation energy via an electroporation device; and sensing the application of the electroporation energy by the electroporation device via an array of sensors to determine a location of the electroporation device within the patient.

In Example 32, the method of Example 31, further comprising indicating the location of the electroporation device within the patient via an output device.

In Example 33, the method of Example 32, wherein indicating the location of the electroporation device occurs during the step of applying the electroporation energy.

In Example 34, the method of Example 31, wherein arranging the array of sensors along an external portion of the patient.

In Example 35, the method of Example 31, wherein sensing the application of the electroporation energy includes measuring an output magnetic field density emitted by the electroporation device over a grid of the array of sensors.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an example electroporation device at a target tissue region within patient's heart in accordance with embodiments of the disclosure.

FIG. 2A shows an example electroporation device and electromagnetic field in accordance with embodiments of the disclosure.

FIG. 2B shows the electroporation device, as shown in FIG. 2A, at a target tissue region prior to application of electroporation energy in accordance with embodiments of the disclosure.

FIG. 2C shows the electroporation device, as shown in FIGS. 2A-B, at a target tissue region after application of electroporation energy in accordance with embodiments of the disclosure.

FIG. 3 shows a partial cross-sectional illustration of an example electroporation device in accordance with embodiments of the disclosure.

FIG. 4 shows an example electroporation system arranged at a target tissue region within patient's heart in accordance with embodiments of the disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

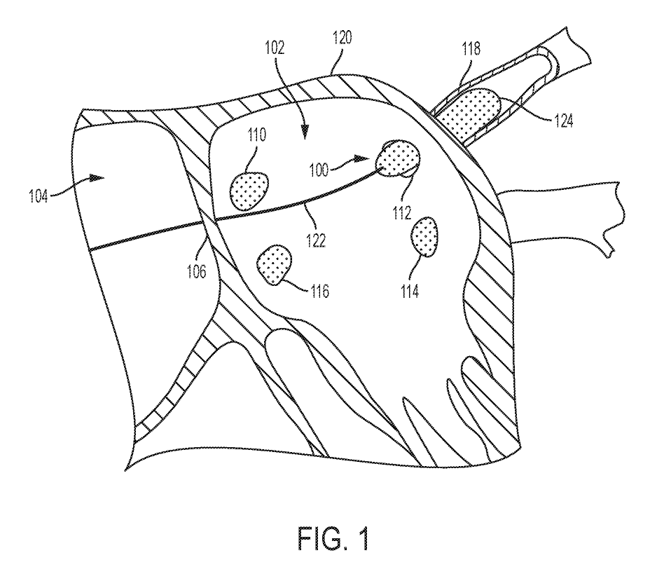

FIG. 1 shows an example electroporation device 100 at a target tissue region within patient's heart 120 in accordance with embodiments of the disclosure. The heart 120 shown in FIG. 1 may be undergoing a pulmonary vein ablation procedure using a electroporation device 100 in accordance with various aspects discussed herein. The electroporation device 100 may be used in other blood vessels or arteries or other portions of the heart such as the left atrial appendage. The electroporation device 100 may include a catheter having an elongate body 122 that is connected to a balloon structure 124. The electroporation device 100 may be connected to an ablation energy source and controller (e.g., radiofrequency (RF) or direct current (DC) system not shown) and one or more liquid sources (not shown), both of which are located external to the patient. The balloon structure 124 may be located near the distal end of elongate body 122. One or more interior chambers of the balloon structure 124 may be in fluid communication with a liquid delivery lumen arranged within the elongate body 122. The liquid delivery lumen is used to convey the one or more liquids from the source external to the patient into the balloon structure 124. The elongate body 122 and the balloon structure 124 may be delivered to a tissue region to which ablation energy may be applied.

As shown in FIG. 1, the elongate body 122 may be positioned in the left atrium 102 of the patient's heart 120. More specifically and in certain instances, the electroporation device 100 may enter the right atrium 104 of heart 120 through a femoral vein and the inferior vena cava (not shown). The electroporation device 100 may be passed through a puncture in an atrial septum 106 to access left atrium 102. From the left atrium 102, the balloon catheter electroporation device 100 may be positioned through any of the pulmonary vein ostia 110, 112, 114, or 116 to enter a pulmonary vein such as pulmonary vein 118. In certain instances, the electroporation device 100 may be an over-the-wire device that is delivered over or on a pre-placed guidewire or a delivery catheter/sheath and/or be self steerable or rapid exchange catheter may be used to assist in the insertion and placement of the electroporation device 100.

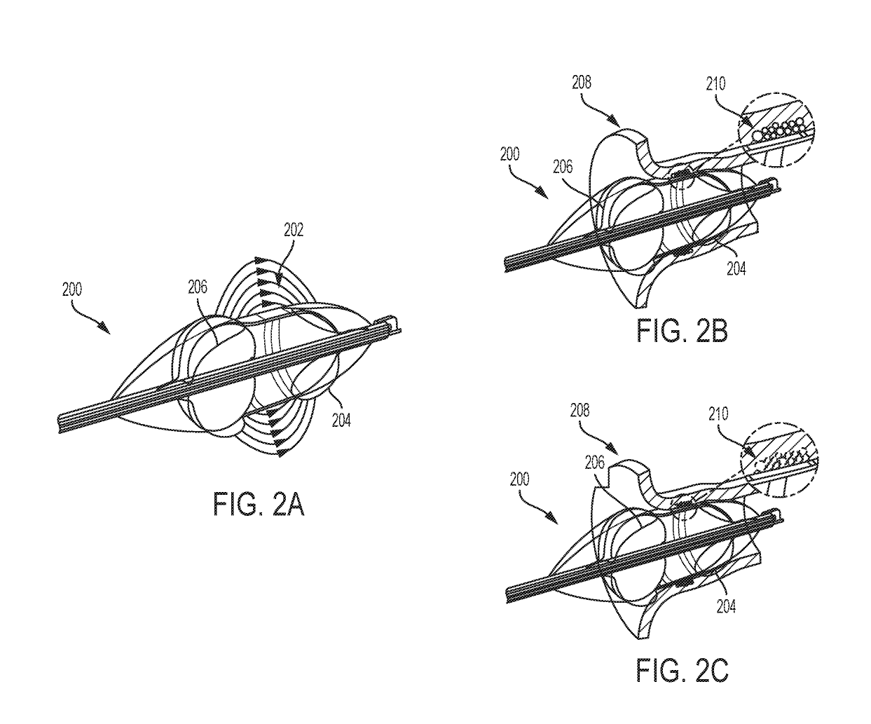

After positioning of the electroporation device 100 at the tissue region (within the pulmonary vein 118 as shown in FIG. 2), the balloon structure 124 may be expanded. The balloon structure 124 may be inflated using a liquid (e.g., saline, a pharmacological agent, or a combination thereof) as the inflation medium. In instances where the balloon structure 124 is positioned within a vessel such as the pulmonary vein 118, the inflation of balloon structure 124 may cause the outer surface of balloon structure 124 to contact an inner wall of vessel such as the pulmonary vein 118. In certain instances, ablation energy may be applied through one or more electrodes (not shown) arranged within the balloon structure 124 to initiate the modulation of target neural fibers. In addition, one or more portions of the balloon structure 124 may have a permeability such that a liquid may exude, elute, weep, or otherwise be transmitted from therethrough. In certain instances, the liquid may be an anti-stenotic pharmaceutical agent that may contact the inner wall of pulmonary vein 118.

The ablation energy may be applied through one or more portions of the balloon structure 124 by an electric field generated by the external source/controller and transferred through wires within one or more lumens of the elongate body 122 to electrodes (not shown) arranged with the balloon structure 124. The electric energy can be transmitted to the inner wall of pulmonary vein 118 directly from the electrodes on the surface of balloon structure 124 or from the electrodes within the balloon structure 124. The electric field may at least partially cause apoptotic cell death and/or non-thermal necrosis to the tissue receiving the ablation energy.

In certain instances, the electric field may be generated by applying direct current to the one or more electrodes arranged within the balloon structure 124. In addition, the use of direct current may cause apoptotic cell death and/or non-thermal necrosis to the tissue receiving the ablation energy. The direct current may form pores in the cells of the wall of the pulmonary vein 118 that are irreversible (e.g., the pores do not close). The balloon structure 124 being in contact with the wall of the pulmonary vein 118 may provide controlled and direct ablation of a target area while mitigating against down-stream proliferation of the ablation energy.

FIG. 2A shows an example electroporation device 200 and electromagnetic field 202 in accordance with embodiments of the disclosure. electroporation device 200 includes a distal electrode 204 and a proximal electrode 206. Current may be applied to the distal electrode 204 and the proximal electrode 206 to create the electromagnetic field 202 between the distal electrode 204 and the proximal electrode 206. In certain instances, direct current may be pulsed from one of the distal electrode 204 and the proximal electrode 206 to the other of the distal electrode 204 and the proximal electrode 206 to create the electromagnetic field 202. The distal electrode 204 and the proximal electrode 206 may be opposite charged to create the electromagnetic field 202. In certain instances, the distal electrode 204 and the proximal electrode 206 are electrically separated or isolated in order to oppositely charge the distal electrode 204 and the proximal electrode 206 to create the electromagnetic field 202.

The electromagnetic field 202 may utilized to verify that the electroporation therapy applied by the electroporation device 200 is delivered at a desired location and/or at a desired effectiveness. Tissue affected by the electromagnetic field 202 may change (e.g., in density) as compared to unaffected tissue.

FIG. 2B shows the electroporation device 200, as shown in FIG. 2A, at a target tissue region 208 prior to application of electroporation energy in accordance with embodiments of the disclosure. As shown in FIG. 2B, a group of cells 210 are highlighted in the target tissue region 208. The group of cells 210 have not been affected (e.g., voided or a ions have not evacuated the group of cells 210) as energy has not been applied to the electroporation device 200 at this point.

FIG. 2C shows the electroporation device 200, as shown in FIGS. 2A-B, at a target tissue region 208 after application of electroporation energy in accordance with embodiments of the disclosure. The same group of cells 210 shown in FIG. 2B are shown as affected as a result of the energy applied to the electroporation device 200. The group of cells 210 may swell or void after electroporation which would change the density of the group of cells 210. The electromagnetic field 202, shown in FIG. 2A, voids or swells the group of cells 210. In comparing the group of cells 210 in FIG. 2B (unaffected cells) and the group of cells 210 in FIG. 2C (affected cells), a difference in density is present therebetween. Local magnetic field variations, or magnetic field gradients, may introduced due to the “voiding” or “swelling” of the impacted cells 210 (shown in FIG. 2C). The localized gradients can be utilized to define a successful application of electroporation energy by the electroporation device 200 within the target tissue region 208.

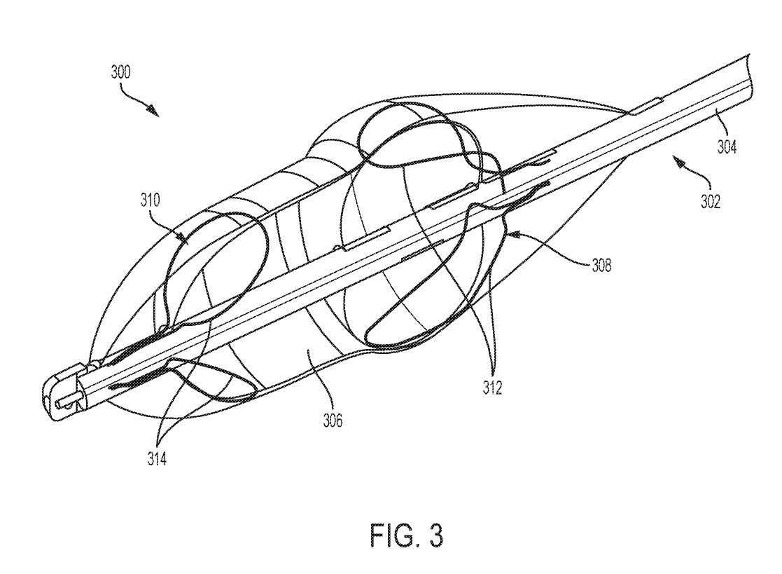

FIG. 3 shows a partial cross-sectional illustration of an example electroporation device 300 in accordance with embodiments of the disclosure. The electroporation device 300 may include a catheter 302 sized and shaped for vascular access that has an elongate body 304 extending between a proximal end and a distal end of the catheter 302. A distal portion of the catheter 302 and the elongate body 304 is shown in FIG. 3. The electroporation device 300 may also include a balloon structure 306 arranged near the distal end of the elongate body 304. The balloon structure 306 may be configured to inflate in response to a liquid or inflation medium being provided thereto.

The electroporation device 300 may also include one or more electrodes arranged on or within the balloon structure 306. As shown in FIG. 3, the apparatus includes a proximal electrode 308 arranged near a proximal end and on or within the balloon structure 306 and a distal electrode 310 arranged near a distal end and on or within the balloon structure 306. The proximal electrode 308 and the distal electrode 310 may be configured to deliver energy to a tissue region. In certain instances, the proximal electrode 308 and the distal electrode 310 may be configured to delivery energy in response to a direct current applied thereto. As a result of the direct current applied to the proximal electrode 308 and the distal electrode 310, an electromagnetic field develops to delivery electroporation energy to the tissue region. The direct current may be applied in pulse bursts applied that results in electroporation energy delivered by the electroporation device 300.

As shown in FIG. 3, the proximal electrode 308 includes portions 312 that extend toward the distal electrode 310 and the distal electrode 310 includes portions 314 that extend toward the proximal electrode 308. These portions 312, 314 may control a shape of the electromagnetic field that develops as a result of the pulse bursts applied to the proximal electrode 308 and the distal electrode 310. In addition, the proximal electrode 308 are spaced apart by between 0 mm and 25 mm.

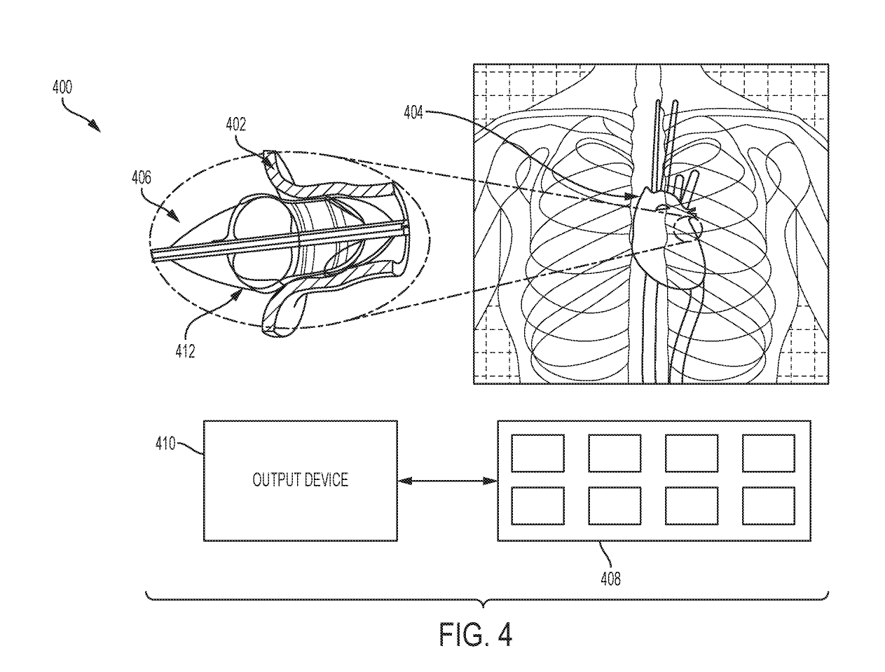

The balloon structure 306 may anchor the electroporation device 300 at a target location within a patient. In certain instances, the target location may be a vein or artery in a patient such as the pulmonary vein. Blood moving through the target location and/or pulsing of the patient's heart may affect movement of the electroporation device 300 and hinder the ability of the electroporation device 300 to accurate delivery electroporation energy. The electroporation device 300 may be used in connection with an array of sensors and an output device as shown in FIG. 4 and discussed in further detail below. The array of sensors and the output device may be configured to verify location of the electroporation device 300 and also may be configured to verify effectiveness of the electroporation energy applied by the electroporation device 300.

FIG. 4 shows an electroporation system 400 at a target tissue region 402 within patient's heart 404 in accordance with embodiments of the disclosure. The electroporation system 400 includes an electroporation device 406, an array of sensors 408, and an output device 410.

The array of sensors 408 are configured to sense an application of electroporation energy by the electroporation device 406 to determine a location of the electroporation device 406 within the patient. In certain instances, the array of sensors 408 are configured to sense an electromagnetic field generated by the electroporation device 406 (e.g., as discussed above with reference to FIGS. 2A-C) resulting from pulse bursts applied by the electroporation device 406. The array of sensors 408 may be a two-dimensional or three-dimensional array of magnoresistive sensors. The array of sensors 408 may be arranged alone one or more sides of the patient. In certain instances, the array of sensors 408 may be arranged beneath a patients back during the electroporation procedure. In addition, the array of sensors 408, when three-dimensional, may extend up at least a portion of the patient's side or sides.

The array of sensors 408 may be magnoresistive sensors such as Magneto-Resistance (AMR) sensors, Giant Magneto-Resistance (GMR) sensors, Magnetic Tunneling Junction (MTJ) sensors, Tunnel Magneto-Resistance (TMR) sensors, inductive sensors, fluxgate sensors, GMI (giant magnetoimpedance) sensors, hall sensors or other similarly configured sensors. In addition, the array of sensors 408 may be configured to measure an output magnetic field density emitted by the electroporation device 406 over a grid of the array of sensors 408.

The array of sensors 408 communicates, wirelessly or via a direct connection, with the output device 410. The output device 410 includes circuitry configured to measure the electromagnetic field generated by the electroporation device 406 and sensed by the array of sensors 408. In certain instances, the output device 410 is configured to measure a difference between in the electromagnetic field generated by the electroporation device 406 prior to application of pulse bursts and the electromagnetic field generated by the electroporation device 406 after to application of the pulse bursts. The difference measured by the output device 410 measures affected cells to determine the location of the electroporation device 406 within the patient. More specifically, the output device 410 is configured to measure the difference based on a change in cell density at the target tissue region 402.

Electroporated cells have a different density that cells that have not been electroporated, and the output device 410 may determine the difference in the electric fields, sensed by the array of sensors 408, of the affected and unaffected cells. Thus, the output device 410 may also be configured to verify effectiveness of the application of electroporation by the electroporation device 406 at the tissue region 402. In certain instances, the output device 410 is configured to measure the change in cell density to locate impacted and non-impacted cells in the tissue region. In addition, the output device 410 may be configured to display an indication of the impacted and non-impacted cells during application of the application of electroporation energy. In this manner, the output device 410 may provide a real-time display of the effectiveness of the electroporation device 406 during application of electroporation by the electroporation device 406 at the tissue region 402.

The electroporation device 406 may also include a balloon structure 412 configured to anchor the electroporation device 406 at the tissue region 402 within the patient. Blood moving through the tissue region and/or pulsing of the patient's heart 404 may affect movement of the electroporation device 406 and hinder the ability of the electroporation device 406 to accurate delivery electroporation energy. The balloon structure 412 may also facilitate application of the electroporation energy to the tissue region 402 by providing a contact area between the electroporation device 406 and the tissue region 402.

In addition, the array of sensors 408 and the output device 410 may provide real-time feedback to an operating physician using the electroporation device 406. The ability of the output device 410 to indicate location of the electroporation device 406 and effectiveness of the electroporation device 406 during use of the electroporation device 406 increases the effectives of the electroporation device 406.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An apparatus for assessing electroporation therapy applied to a tissue region of a patient, the apparatus comprising:
an array of sensors configured to sense an electromagnetic field generated during the application of electroporation energy by an electroporation device to determine a location of the electroporation device within the patient; and
an output device configured to indicate the location of the electroporation device within the patient.

2. The apparatus of claim 1, wherein the output device is configured to verify non-movement of the electroporation device between pulse bursts of the electroporation device.

3. The apparatus of claim 1, wherein the output device is configured to measure a difference between in the electromagnetic field generated by the electroporation device prior to application of the pulse bursts and the electromagnetic field generated by the electroporation device after to application of the pulse bursts effective cells to determine the location of the electroporation device within the patient.

4. The apparatus of claim 3, wherein the output device is configured to measure the difference based on a change in cell density at the tissue region.

5. The apparatus of claim 3, wherein the output device is configured to measure the change in cell density to locate impacted and non-impacted cells in the tissue region.

6. The apparatus of claim 5, wherein the output device is configured to display an indication of the impacted and non-impacted cells during application of the application of electroporation energy.

7. The apparatus of claim 1, wherein output device is further configured to verify effectiveness of the application of electroporation energy by the electroporation device at the tissue region.

8. The apparatus of claim 1, wherein the electroporation device includes a balloon structure and one or more electrodes arranged on or within the balloon structure and configured to deliver energy to the tissue region.

9. An apparatus for assessing electroporation therapy applied to a tissue region, the apparatus comprising:
an electroporation device comprising:
a catheter sized and shaped for vascular access and including an elongate body extending between a proximal end and a distal end,
a balloon structure arranged near the distal end of the elongate body, and
one or more electrodes arranged on or within the balloon structure and configured to deliver energy to the tissue region; and
an array of sensors configured to sense an application of electroporation energy by the electroporation device to determine a location of the electroporation device within the patient.

10. The apparatus of claim 9, wherein the array of sensors are at least one of a two-dimension array of magnoresistive sensors and three-dimensional array of magnoresistive sensors.

11. The apparatus of claim 10, wherein the magnoresistive sensors include at least one of Magneto-Resistance (AMR) sensors, Giant Magneto-Resistance (GMR) sensors, Magnetic Tunneling Junction (MTJ) sensors, and Tunnel Magneto-Resistance (TMR) sensors, inductive sensors, fluxgate sensors, GMI (giant magnetoimpedance) sensors, and hall sensors.

12. The apparatus of claim 9, wherein the array of sensors are configured to measure an output magnetic field density emitted by the electroporation device over a grid of the array of sensors.

13. The apparatus of claim 9, wherein the one or more electrodes includes a proximal electrode arranged near a proximal end of the balloon structure and a distal electrode arranged near a distal end of the balloon structure, and the proximal electrode includes portions extending toward the distal electrode and the distal electrode includes portions extending toward the proximal electrode.

14. The apparatus of claim 13, wherein the proximal electrode and the distal electrode are spaced apart by between 0 mm and 25 mm.

15. The apparatus of claim 9, wherein output device is further configured to verify effectiveness of the application of electroporation energy by the electroporation device at the tissue region.

16. A method of assessing electroporation therapy applied to a tissue region of a patient, the method comprising:
applying electroporation energy via an electroporation device; and
sensing the application of the electroporation energy by the electroporation device via an array of sensors to determine a location of the electroporation device within the patient.

17. The method of claim 16, further comprising indicating the location of the electroporation device within the patient via an output device.

18. The method of claim 17, wherein indicating the location of the electroporation device occurs during the step of applying the electroporation energy.

19. The method of claim 16, wherein arranging the array of sensors along an external portion of the patient.

20. The method of claim 16, wherein sensing the application of the electroporation energy includes measuring an output magnetic field density emitted by the electroporation device over a grid of the array of sensors.
